(19) United States
(12) Patent Application Publication
(10) Pub. No.: US 2024/0108850 A1
(43) Pub. Date: Apr. 4, 2024

(54) Urinary Catheter

(71) Applicant: C. R. Bard, Inc., Franklin Lakes, NJ (US)

(72) Inventors: Zhihui Yin, Lilburn, GA (US); David Fish, Rutledge, GA (US); Tom Roberts, Bishop, GA (US)

(21) Appl. No.: 18/536,063

(22) Filed: Dec. 11, 2023

Related U.S. Application Data
(63) Continuation of application No. 17/114,275, filed on Dec. 7, 2020, now Pat. No. 11,850,370.
Application No. 17/114,275 is a continuation of application No. 15/506,723, filed on Feb. 24, 2017, now Pat. No. 10,857,324.
(60) Provisional application No. 62/042,125, filed on Aug. 26, 2014.

(51) Int. Cl. A61M 25/00; A61L 29/08; A61L 29/14; A61M 25/01

(52) U.S. Cl. CPC A61M 25/0017; A61L 29/085; A61L 29/14; A61L 29/145; A61M 25/002; A61M 25/0045; A61M 25/007; A61M 25/0136; A61L 29/08; A61L 29/141; A61L 2400/10; A61L 2420/08; A61M 25/0009; A61M 2025/0046

(57) ABSTRACT

A urinary catheter and container. The urinary catheter can include a catheter shaft attached to a handle and a coating disposed on an outer surface of the catheter shaft. The coating can include a hydrogel, water and/or glycerin, and a polyethylene gylcol (PEG). The PEG can have a molecular weight equal to or less than 600, for example one or more of polyethylene glycol (PEG) 300 and PEG 400. The coating can be applied in a wet state and remain wet for an extended period of time in the container, thereby obviating the need for a lubricant, such as a water sachet or gel package, to accompany the catheter in the container. The container can include a gas impermeable foil material. The container can include an adhesive tab covering a perforated section, the adhesive tab including a pull loop.

### PRIORITY

This application is a continuation of U.S. patent application Ser. No. 17/114,275, filed Dec. 7, 2020, which is a continuation of U.S. patent application Ser. No. 15/506,723, a U.S. national stage application under 35 U.S.C. § 371 of International Application No. PCT/US2015/047026, filed Aug. 26, 2015, now U.S. Pat. No. 10,857,324, which claims the benefit of priority to U.S. Provisional Application No. 62/042,125, filed Aug. 26, 2014, each of which is incorporated by reference in its entirety into this application.

### BACKGROUND

People suffering from neurogenic bladder disorders like spinal cord injury, spina bifida or multiple sclerosis, and non-neurogenic bladder disorders like obstruction due to prostate enlargement, urethral strictures or post-operative urinary retention, need to be continuously catheterized to empty their urinary bladders. However, continuous catheterization can lead to problems like urinary tract infections (UTI), urethral strictures or male infertility. Intermittent catheterization at regular intervals avoids many of the negative effects of continuous long term catheterization. There are four primary categories for intermittent catheters: (1) Bare Intermittents, (2) Hydrophilic Coated Intermittents, (3) Pre-Wetted Intermittents, and (4) Catheter in Bag or “Touchless” Intermittents.

Bare Intermittents require the use of an external lubrication method. These catheters are the least expensive and most commonly used. Typical materials include natural rubber (latex) (NRL), polyvinyl chloride (PVC) and silicone. The common lubrication method is a gel pack. The gel is either applied to the meatus of the urethra or the tip of the catheter itself. Hydrophilic Coated Intermittents have a lubricious coating applied typically to the first two-thirds of the shaft of the catheter and are activated by breaking a water sachet located inside the package prior to opening the package. When activated, the catheter is lubricious for insertion into the urethra. Potential issues with the Bare Intermittents and the Hydrophilic Coated Intermittents include the amount of mess they create (e.g., from the excess water from the water sachet and lubricant from the lubricant packs) and the time required for the user to complete the voiding process.

Pre-Wetted Intermittents may be packaged in a non-permeable package (e.g. foil, or rigid plastic) and suspended in water. Ideally, the catheters will stay wet over the length of their shelf life and may be much like hydrophilic coated intermittents that have been activated by water. Pre-Wetted Intermittents may have a lubricious coating in addition to being packaged in water. This can eliminate the process step of lubricating the catheter, but may still some mess to contend with (e.g., from the water stored in the package), and the coating may dry out over its shelf life making it unusable.

Catheter in Bag or “Touchless” Intermittents may include either a Bare Intermittent or Hydrophilic Coated Intermittent. There may be an insertion tip on an end of the bag with the distal end of the catheter captured in the insertion tip. Upon use, the user may advance the catheter out of the bag using the insertion tip to help guide the catheter into the urethra. The bag may be used for urine collection. However, use of a Touchless Catheter may be cumbersome and difficult.

The following are references relating to coatings: U.S. Pat. Nos. 6,673,053, 8,011,505, and 6,059,107, which are incorporated by reference herein in their entireties.

### SUMMARY

The urinary catheters described herein provide a novel type of intermittent catheter not currently available. The coating may exhibit hygroscopic characteristics, described herein as the characteristic or intention of the coating to not only retain the moisture inherent in the coating but also to attract moisture from the environment. The coating may exhibit hydrophilic characteristics. The coating described herein is an improved formulation that is applied in a wet state and stays wet for an extended period of time. Accordingly, the urinary catheters described herein do not require an additional lubricant or wetting component, such as a water sachet or gel package, to accompany the catheters in the containers. The urinary catheters described herein may be packaged individually in a discrete container, such as an opaque foil. These and other features of embodiments of the present invention will become more fully apparent from the following description and appended claims, or may be learned by the practice of embodiments of the invention as set forth hereinafter.

In one embodiment a urinary catheter may include a catheter shaft attached to a handle. The urinary catheter may also include a hygroscopic and/or hydrophilic coating disposed on an outer surface of the catheter shaft. The coating may include a hydrogel, glycerin or water, and a polyethylene glycol (PEG). In one embodiment, the hydrogel may be LUBRAJEL® RR CG hydrogel or LUBRAJEL® RR hydrogel, and the PEG may be one or both of PEG 300 and PEG 400. In embodiments described herein with respect to specific hydrogels (e.g., LUBRAJEL® RR CG hydrogel), other hydrogels (e.g., LUBRAJEL® RR hydrogel) are contemplated as being substituted for, or added to, the specified hydrogel. Likewise, in embodiments described herein with respect to specific polyethylene glycols (e.g., PEG 300), other polyethylene glycols are contemplated as being substituted for, or added to, the specified polyethylene glycol.

In one embodiment, a urinary catheter includes a catheter shaft attached to a handle, and a first coating disposed on an outer surface of the catheter shaft, the first coating including a hydrogel or polyacrylic acid (PAA), glycerin and/or water, and polyethylene glycol (PEG), the first coating exhibiting hygroscopic and/or hydrophilic characteristics. In one embodiment, the outer surface of the catheter shaft includes a second coating over which the first coating is disposed. In one embodiment, the second coating is a hydrophilic coating.

In one embodiment, the coating formulations described herein provide non-adhesion (or anti-blocking) toward the packaging material. In one embodiment, a catheter with the coating can be sterilized through electron beam (“e-beam”) sterilization or ethylene oxide (EtO) sterilization. In one embodiment, an additional ultraviolet (UV)-curable silicone film can be applied over a catheter with the coating described herein. The silicone film may restrict the coating on the catheter. In one embodiment, the film may be moved, e.g., toward the catheter handle, thereby acting as a touchless layer while maintaining the lubricity of the catheter. In one embodiment, the UV-curable silicone film is disposed on the coating via an UV curing process.

In one embodiment of the packaged urinary catheter, a coating formulation (e.g., a formulation for a base coating and/or outer coating) for the catheter may include LUBRAJEL® RR CG hydrogel in a range of 15 wt % to 35 wt %, water in a range of 10 wt % to 45 wt %, and PEG in a range of 20 wt % to 75 wt %. In one embodiment, a coating formulation may include LUBRAJEL® RR CG hydrogel in a range of 20 wt % to 30 wt %, water in a range of 20 wt % to 30 wt %, and PEG 400 in a range of 40 wt % to 60 wt %. In one embodiment, the coating formulation may include LUBRAJEL® RR CG hydrogel in a range of 22 wt % to 26 wt %, water 25 wt %, and PEG 400 in a range of 49 wt % to 53 wt %. In one embodiment the coating formulation may include LUBRAJEL® RR CG hydrogel at 23.5 wt %, water at 25 wt %, and PEG 400 at 51.5 wt %. In one embodiment, a coating formulation may include LUBRAJEL® RR CG hydrogel in a range of 20 wt % to 30 wt %, glycerin in a range of 20 wt % to 30 wt %, and PEG 400 in a range of 40 wt % to 60 wt %. In one embodiment, the coating formulation may include LUBRAJEL® RR CG hydrogel in a range of 20 wt % to 30 wt %, glycerin in a range of 40 wt % to 60 wt %, and PEG 300 in a range of 20 wt % to 30 wt %. In one embodiment, the coating formulation may include LUBRAJEL® RR CG hydrogel in a range of 10 wt % to 35 wt %, glycerin in a range of 25 wt % to 75 wt %, PEG 300 in a range of 25 wt % to 65 wt %, and PEG 400 in a range of 25 wt % to 50 wt %. In one embodiment, a coating formulation may include LUBRAJEL® RR CG hydrogel in a range of 20 wt % to 30 wt %, glycerin in a range of 40 wt % to 60 wt %, propylene glycol (PEG) in a range of 10 wt % to 15 wt %, and ethanol (anhydrous) in a range of 10 wt % to 15 wt %. In one embodiment, the LUBRAJEL® RR CG hydrogel is 50 wt %, the glycerin is 25 wt %, and both the PEG and ethanol are 12.5 wt %.

In one embodiment, a coating formulation may include LUBRAJEL® RR hydrogel in a range of 15 wt % to 35 wt %, glycerin in a range of 15 wt % to 30 wt %, and PEG 400 in a range of 35 wt % to 70 wt %. In one embodiment, the coating formulation may include LUBRAJEL® RR hydrogel at 25 wt %, glycerin at 25 wt %, and both PEG 300 and PEG 400 at 25 wt %. In one embodiment, the coating formulation may include LUBRAJEL® RR hydrogel at 40 wt %, glycerin at 15 wt %, PEG 300 at 15 wt %, and PEG 400 at 30 wt %. In one embodiment, a coating formulation may include LUBRAJEL® RR in a range of 20 wt % to 30 wt %, water in a range of 20 wt % to 30 wt %, and PEG 400 in a range of 40 wt % to 60 wt %.

In one embodiment, a coating formulation may include polyacrylic acid (PAA) in a range of 0.2 wt % to 3 wt %, glycerin in a range of 15 wt % to 25 wt %, water in a range of 20 wt % to 30 wt %, and PEG 400 in a range of 40 wt % to 60 wt %. In one embodiment, a coating formulation may include PAA in a range of 0.1 wt % to 2.5 wt %, water in a range of 10 wt % to 45 wt % and PEG, such as PEG 300 and/or PEG 400, in a range of 20 wt % to 65 wt %.

In one embodiment, a silicone film may be formed over a coating on a catheter. In one embodiment, a method of forming a catheter with a coating includes dipping a coated catheter, such as a hydrophilic coated catheter, into a solution containing any of the coating formulations herein, such as a coating formulation including PAA, water, and PEG or a coating formulation including hydrogel, glycerin and/or water, and PEG, then dipping the twice-coated catheter into a UV curable solution, then exposing the coated areas to a UV source, and then directly placing the catheter into a package. In one embodiment, the hydrophilic coated catheter is dipped into a PAA/water/PEG solution for a dwell time in a range of 0.1 seconds to 10 seconds. In one embodiment, after the catheter is dipped into the PAA/water/PEG solution, it is dipped into a silicone solution with UV curable agents several times to achieve a desired film thickness. In one embodiment, the desired thickness is 0.001 in. to 0.004 in. In one embodiment, the catheter is dipped into the silicone solution with UV curable agents 2 to 6 times. In one embodiment, after being dipped into the silicone solution with UV curable agents, the catheter is exposed to a UV source, such as a UV light, in a time range of 0.3 min to 2.0 min. In one embodiment, following the exposure to the UV source, the catheter is placed directly into a film, foil, and/or Tyvek package without a further drying process.

In one embodiment, a method of making a urinary catheter includes applying a first coating to a catheter shaft, the first coating comprising a hydrogel or polyacrylic acid (PAA), glycerin and/or water, and polyethylene glycol (PEG) to form a coated catheter, and placing the coated catheter into a package comprising a gas impermeable foil material. In one embodiment, the catheter shaft includes a base hydrophilic coating, and the first coating is applied over the base hydrophilic coating. In one embodiment, the applying includes dipping the catheter shaft with the base hydrophilic coating into a solution containing a formulation of the first coating. In one embodiment, the first coating formulation comprises only the PAA, the water, and the PEG, further comprising dipping the coated catheter into a silicone solution including ultraviolet (UV) curable agents to form a silicone film over the first coating. In one embodiment, the method includes exposing the silicone film to a UV light source for a period of time to cure the silicone solution.

In one embodiment, the urinary catheter may include an eyelet or a plurality of staggered, opposing eyelets (e.g., 3, 4, 5, 6, 7, 8, or more eyelets) proximal to a catheter tip, the eyelets may be arranged in a variety of ways, including circumferentially positioned 90 degrees apart and positioned in a non-overlapping configuration. In one embodiment, the urinary catheter shaft includes a funnel shaped proximal end and ridges configured to facilitate gripping. In one embodiment, the urinary catheter may have a coating that exhibits hygroscopic characteristics. In another embodiment, the urinary catheter may have a coating that exhibits hydrophilic characteristics.

In one embodiment, a packaged urinary catheter may include a container and a urinary catheter. The urinary catheter may include a catheter shaft attached to a handle and a coating disposed on an outer surface of the catheter shaft. In one embodiment, the coating may include a hydrogel, glycerin or water, and PEG, such as one or both of PEG 300 and PEG 400. In one embodiment, the coating may include PAA, glycerin, water, and PEG, such as PEG 300 and/or PEG 400. In one embodiment, the coating may include PAA, water, and PEG, such as PEG 300 and/or PEG 400.

In one embodiment of the packaged urinary catheter, the container may include a gas impermeable foil material. In one embodiment of the packaged urinary catheter, the container may include an adhesive tab covering a perforated section of the foil material, the adhesive tab may include a pull loop. In one embodiment, the container may include a water sachet, gel package, or other type of lubricant therein. In one embodiment, the container may include a moisture source (in contact or separated from the catheter) from which a hygroscopic coating and/or a hydrophilic coating on the urinary catheter may absorb or obtain moisture. In one embodiment of the packaged urinary catheter, the container does not include any water sachet, gel package, or other type of lubricant or moisture source therein.

In one embodiment, a method of catheterizing may include obtaining a urinary catheter that may include a handle and a catheter shaft. The catheter shaft may include a hydrophilic coating and/or a hygroscopic coating on an outer surface thereof. In one embodiment, the coating may include a coating formulation described herein. The method may further include inserting the urinary catheter into a bladder. In one embodiment, the method of catheterizing may include obtaining the urinary catheter from a container in which the urinary catheter has been stored. In one embodiment, the method of catheterizing does not include application of a lubricant or water to the catheter shaft at any time prior to insertion into the bladder, including while in the package.

### BRIEF DESCRIPTION OF THE DRAWINGS

The disclosed systems and methods can be better understood with reference to the following drawings. The components in the drawings are not necessarily to scale. It is appreciated that these drawings depict only typical embodiments of the invention and are therefore not to be considered limiting of its scope. Example embodiments of the invention will be described and explained with additional specificity and detail through the use of the accompanying drawings in which:

FIG. 1 shows one embodiment of a urinary catheter according to embodiments described herein, and illustrates the exemplary use of a male urinary catheter according to embodiments described herein.

FIG. 2 shows one embodiment of a urinary catheter according to embodiments described herein, and illustrates the exemplary use of a female urinary catheter according to embodiments described herein.

FIG. 3a is a urinary catheter according to embodiments described herein.

FIG. 3b is a cross sectional view of the urinary catheter shaft according to embodiments described herein.

FIG. 4a is a first step in a method of making the container for a urinary catheter according to embodiments described herein.

FIG. 4b is a second step in a method of making the container for a urinary catheter according to embodiments described herein.

FIG. 4c is a third step in a method of making the container for a urinary catheter according to embodiments described herein.

FIG. 5 is a container for a urinary catheter of FIGS. 4-7, according to embodiments described herein in a closed state.

FIG. 6 is the container for a urinary catheter of FIGS. 4-7, being opened according to embodiments described herein.

FIG. 7 is the container of FIGS. 4-7 in an opened state, revealing the urinary catheter handle.

While the invention is susceptible to various modifications and alternative forms, specific embodiments thereof have been shown by way of example in the drawings and are herein described in detail. It should be understood, however, that the description herein of specific embodiments is not intended to limit the invention to the particular forms disclosed, but rather the intention is to cover all modifications, equivalents, and alternatives falling within the spirit and scope of the invention as defined by the appended claims.

### DETAILED DESCRIPTION

The following description and accompanying figures, which describe and show certain embodiments, are made to demonstrate, in a non-limiting manner, several possible configurations of a catheter according to various aspects and features of the present disclosure. While the description herein, by way of example, is focused primarily on a description of a urinary catheter and associated methods, the inventions described herein are not so limited and the concepts may be applied to other types of catheters and devices.

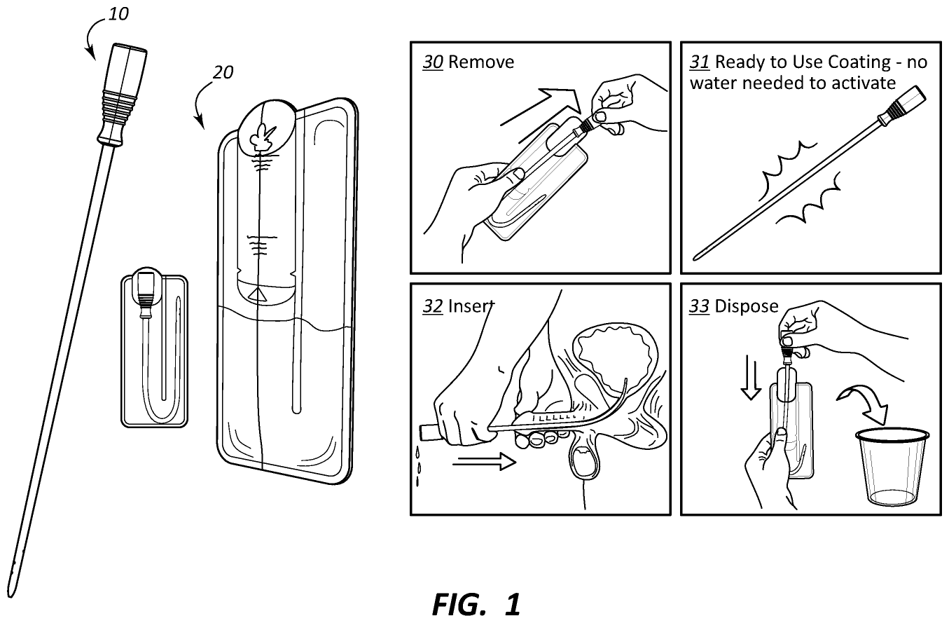

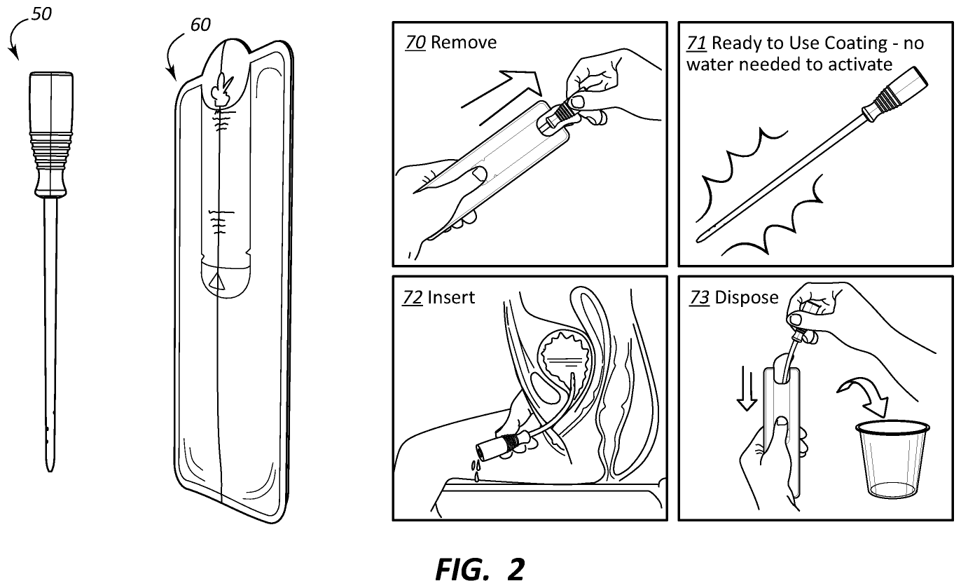

The urinary catheter described herein is ready to use immediately when the container is opened, and may be inserted by the patient or patient's caregiver in a homecare setting, managed care/assisted living setting, or in hospitals. Within the homecare setting, the catheter can be used in a range of restroom and non-restroom environments. FIGS. 1 and 2 show urinary catheters and methods of using them according to embodiments described herein.

FIG. 1 illustrates the male urinary catheter 10, the packaging 20 for the male urinary catheter 10, and the exemplary use (e.g., steps 30-33) thereof according to embodiments described herein, and FIG. 2 illustrates the female urinary catheter 50, the packaging 60 for the female urinary catheter 50, and the exemplary use (e.g., steps 70-73) thereof according to embodiments described herein. The methods shown in FIGS. 1 and 2 do not require the user to take any step to apply lubricant, such as water or gel, directly to the catheter, either while the catheter is within the package or when after the package has been opened. Accordingly, the user may move directly from the step of removing the catheter from the package 30, 70 to the step of inserting the catheter 32, 72 without an intervening direct lubrication or hydration step (see example steps 31, 71, which indicate the catheter is ready to use upon removing from the packaging, without requiring the addition of water or lubricant). The catheters used in FIGS. 1 and 2 can be catheters of any of the embodiments discussed herein, e.g., the catheters may have a coating formulation that exhibits hygroscopic and/or hydrophilic characteristics (which eliminates the need for the user to take steps to lubricate or hydrate the catheter). In the case of a catheter with a hygroscopic coating, while some water from the surrounding environment may be naturally attracted by the coating, this is not considered a direct lubrication or hydration step taken in the method. After use, the catheter 10, 50 may be disposed of according to sanitary procedure. Example disposal steps 33, 73 depict one possible procedure for disposal, including returning the catheter to the packaging and discarding the packaging in a trash can or similar receptacle. The packaging may be sealable (e.g., by adhesive, zip-lock, etc.), such that the package may be sealed shut after the urinary catheter is disposed therein.

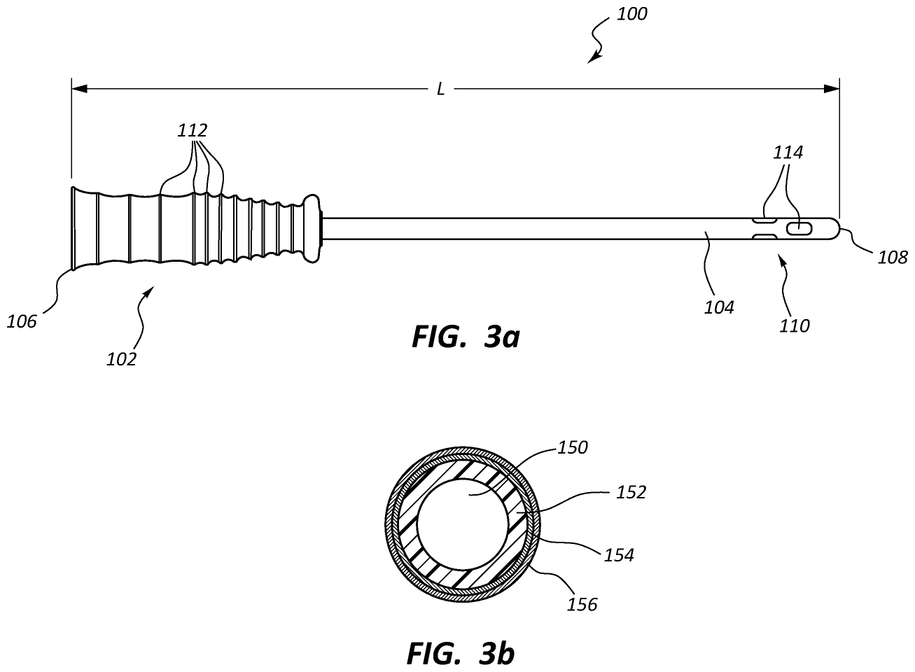

Referring to FIG. 3a, in one embodiment, a urinary catheter 100 includes a handle 102 on a proximal end and a catheter shaft 104 attached to the handle 102. The urinary catheter may be one of a variety of different types of urinary catheters. The handle 102 may have a funnel-like shape 106 on the proximal end thereof, and may be adapted to connect to drain bags, extension tubes, and/or the like. Also, handle shapes other than a funnel-like shape may be utilized within the scope of the present disclosure. The handle 102 may indicate the size of the catheter, and may have a color to indicate sex (e.g., pink for female, blue for male). In one embodiment, the catheter shaft 104 is made from a silicone material. In one embodiment, the silicone material has a durometer in the range of shore 70 A to 85 A and a thickness in the range of 1.1 mm to 2.27 mm. It is appreciated that the composition of the catheter shaft 104 may include other materials that possess similar physical properties which falls within scope of the present disclosure. In one embodiment, the column strength of the catheter shaft 104 is configured or designed to facilitate insertion, e.g., requiring less force than current polyvinyl chloride (PVC) catheters. In one embodiment, the catheter 100 will be at least partially transparent to an unaided eye.

Referring to FIGS. 3a and 3b, the catheter 100 includes openings 114 in a distal end 110 that are in fluid communication with a lumen 150 that extends through the catheter shaft and handle. In one embodiment, the catheter includes four staggered, opposing eyelets 114 proximal to a catheter tip 108, the eyelets 114 are circumferentially positioned 90 degrees apart and positioned in a non-overlapping configuration. It is appreciated that other numbers and configurations of openings fall within the scope of the present disclosure. The handle 102 includes ridges 112 to provide a gripping surface for easier gripping and handling. The catheter shaft 104 may include the lumen 150, a catheter wall 152, a hydrophilic base coating 154 (e.g., polyacrylic acid), and may also include a pre-hydrated outer coating applied thereover 156 (e.g., over the base coating). The pre-hydrated coating may remain wet without the application of water or lubricant gel.

In one embodiment, the catheter 100 includes a hygroscopic coating 156 (e.g. a top or outer pre-hydrated coating). In one embodiment, the catheter 100 includes a hygroscopic coating 156 including a hydrogel, glycerin, water, and a polyethylene gylcol (PEG) with a molecular weight equal to or less than 600, for example one or more of polyethylene glycol (PEG) 300 and PEG 400. In one embodiment, the hydrogel is a LUBRAJEL® hydrogel. For coating embodiments described herein, the type of LUBRAJEL® hydrogel may be LUBRAJEL® RR CG hydrogel, having an INCI name of Glycerin (and) Glyceryl Acrylate/Acrylic Acid Copolymer (and) Propylene Glycol. For coating embodiments described herein, the type of LUBRAJEL® hydrogel may be LUBRAJEL® RR hydrogel. In one embodiment, the catheter includes a coating including a hydrogel (e.g., LUBRAJEL® hydrogel), glycerin, propylene glycol (PEG), and ethanol. In one embodiment, the catheter includes a coating including a hydrogel (e.g., LUBRAJEL® hydrogel), glycerin or water, and propylene glycol (PEG), such as PEG 300 and/or PEG 400. In one embodiment, the catheter may be sold and packaged in sizes ranging in diameter from 8 Fr to 24 Fr (e.g., 8 Fr, 10 Fr, 12 Fr, 14 Fr, 16 Fr, 18 Fr, 20 Fr, 22 Fr, 24 Fr) with a length L of greater than 155 mm and intended for female use. However, other sizes of catheters may also be used. In other embodiments, the catheter may be sold and packaged in various sizes for male use.

In one embodiment, the base coating 154 and/or the outer coating 156 may be applied to the catheter shaft by a method involving either dipping, brushing, spraying or extruding. It is appreciated that other methods of applying one or both of the coatings to the catheter may be utilized and fall within the scope of the present disclosure. In one embodiment, the catheter shaft may be dipped into a volume of coating formulation. In one embodiment, the components of the coating formulation are mixed together, then the catheter shaft dipped into the volume thereof. For example, the hydrophilic coating or outer coating may be produced by mixing LUBRAJEL® with water and PEG for between 1.5 to 4.0 hours. The catheter (with or without a base coating) may be dipped into the coating solution and left to dwell for between 0.1-10 seconds. The catheter may then be removed from the coating solution and directly placed into packaging without any further drying process.

In one embodiment, the eyelets are punched into the catheter prior to dipping into one or more coating formulations to form a coating (e.g., a base coating and/or outer coating) such that both interior and exterior of the catheter is coated, i.e., at least a portion of the outer surface of the catheter shaft and at least a portion of the inner wall defining the lumen 150 of the catheter shaft are coated with the coating formulation. In other embodiments, one or more coating formulations may be brushed onto an outer surface of the catheter shaft (e.g., doctor blade method). In one embodiment, the coating (e.g., the base coating and/or the outer coating) is only on the catheter shaft (either the entire catheter shaft or a distal portion thereof), not on the handle. The coating described herein provides the urinary catheter with a coefficient of friction (COF) in the range of 0.03 to 0.15.

In one embodiment, a coating formulation (e.g., a formulation for a base coating and/or outer coating) for the catheter may include LUBRAJEL® RR CG hydrogel in a range of 15 wt % to 35 wt %, water in a range of 10 wt % to 45 wt %, and PEG in a range of 20 wt % to 75 wt %. In one embodiment, a coating formulation may include LUBRAJEL® RR CG hydrogel in a range of 15 wt % to 35 wt %, water in a range of 2 wt % to 45 wt %, and PEG in a range of 20 wt % to 75 wt %. In one embodiment, a coating formulation may include LUBRAJEL® RR CG hydrogel in a range of 20 wt % to 30 wt %, water in a range of 20 wt % to 30 wt %, and PEG 400 in a range of 40 wt % to 60 wt %. In one embodiment, the coating formulation may include LUBRAJEL® RR CG hydrogel in a range of 22 wt % to 26 wt %, water 25 wt %, and PEG 400 in a range of 49 wt % to 53 wt %. In one embodiment the coating formulation may include LUBRAJEL® RR CG hydrogel at 23.5 wt %, water at 25 wt %, and PEG 400 at 51.5 wt %. In one embodiment, a coating formulation may include LUBRAJEL® RR CG hydrogel in a range of 20 wt % to 30 wt %, glycerin in a range of 20 wt % to 30 wt %, and PEG 400 in a range of 40 wt % to 60 wt %. In one embodiment, the coating formulation may include LUBRAJEL® RR CG hydrogel in a range of 20 wt % to 30 wt %, glycerin in a range of 40 wt % to 60 wt %, and PEG 300 in a range of 20 wt % to 30 wt %. In one embodiment, the coating formulation may include LUBRAJEL® RR CG hydrogel in a range of 10 wt % to 35 wt %, glycerin in a range of 25 wt % to 75 wt %, PEG 300 in a range of 25 wt % to 65 wt %, and PEG 400 in a range of 25 wt % to 50 wt %. In one embodiment, a coating formulation may include LUBRAJEL® RR CG hydrogel in a range of 20 wt % to 30 wt %, glycerin in a range of 40 wt % to 60 wt %, propylene glycol (PEG) in a range of 10 wt % to 15 wt %, and ethanol (anhydrous) in a range of 10 wt % to 15 wt %. In one embodiment, the LUBRAJEL® RR CG hydrogel is 50 wt %, the glycerin is 25 wt %, and both the PEG and ethanol are 12.5 wt %.

In one embodiment, a coating formulation may include LUBRAJEL® RR hydrogel in a range of 15 wt % to 35 wt %, glycerin in a range of 15 wt % to 30 wt %, and PEG 400 in a range of 35 wt % to 70 wt %. In one embodiment, the coating formulation may include LUBRAJEL® RR hydrogel at 25 wt %, glycerin at 25 wt %, and both PEG 300 and PEG 400 at 25 wt %. In one embodiment, the coating formulation may include LUBRAJEL® RR hydrogel at 40 wt %, glycerin at 15 wt %, PEG 300 at 15 wt %, and PEG 400 at 30 wt %. In one embodiment, a coating formulation may include LUBRAJEL® RR in a range of 20 wt % to 30 wt %, water in a range of 20 wt % to 30 wt %, and PEG 400 in a range of 40 wt % to 60 wt %.

In one embodiment, a coating formulation may include polyacrylic acid (PAA) in a range of 0.2 wt % to 3 wt %, glycerin in a range of 15 wt % to 25 wt %, water in a range of 20 wt % to 30 wt %, and PEG 400 in a range of 40 wt % to 60 wt %. In one embodiment, a coating formulation may include PAA in a range of 0.1 wt % to 2.5 wt %, water in a range of 10 wt % to 45 wt % and PEG, such as PEG 300 and/or PEG 400, in a range of 20 wt % to 65 wt %.

In one embodiment, a silicone film may be formed over a coating on a catheter. In one embodiment, a method of forming a catheter with a coating includes dipping a coated catheter, such as a hydrophilic coated catheter, into a solution containing any of the coating formulations herein, such as a coating formulation including PAA, water, and PEG or a coating formulation including hydrogel, glycerin and/or water, and PEG, then dipping the twice-coated catheter into a UV curable solution, then exposing the coated areas to a UV source, and then directly placing the catheter into a package. In one embodiment, the hydrophilic coated catheter is dipped into a PAA/water/PEG solution for a dwell time in a range of 0.1 seconds to 10 seconds. In one embodiment, after the catheter is dipped into the PAA/water/PEG solution, it is dipped into a silicone solution with UV curable agents several times to achieve a desired film thickness. In one embodiment, the desired thickness is 0.001 in. to 0.004 in. In one embodiment, the catheter is dipped into the silicone solution with UV curable agents 2 to 6 times. In one embodiment, after being dipped into the silicone solution with UV curable agents, the catheter is exposed to a UV source, such as a UV light, in a time range of 0.3 min to 2.0 min. In one embodiment, following the exposure to the UV source, the catheter is placed directly into a film, foil, and/or Tyvek package without a further drying process. The silicone with UV curable agents, after curing forms a film that covers the coating on the catheter and can be moved when the catheter is ready for insertion. This acts to facilitate insertion without touching the lubricious coating while maintaining the lubricity of the coating on the catheter.

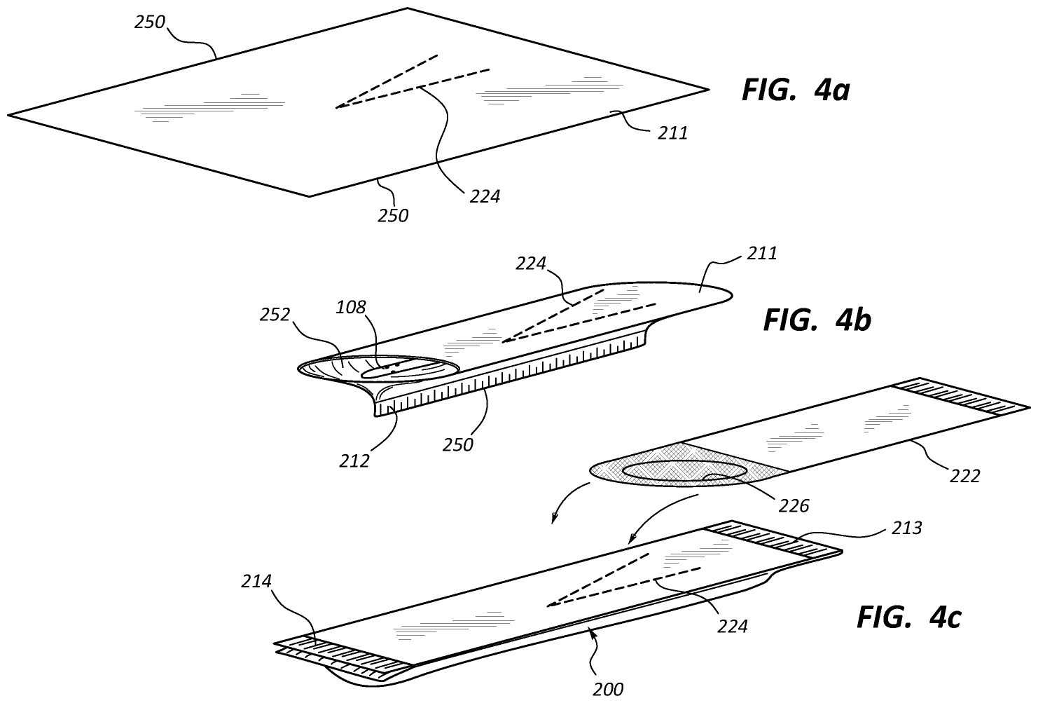

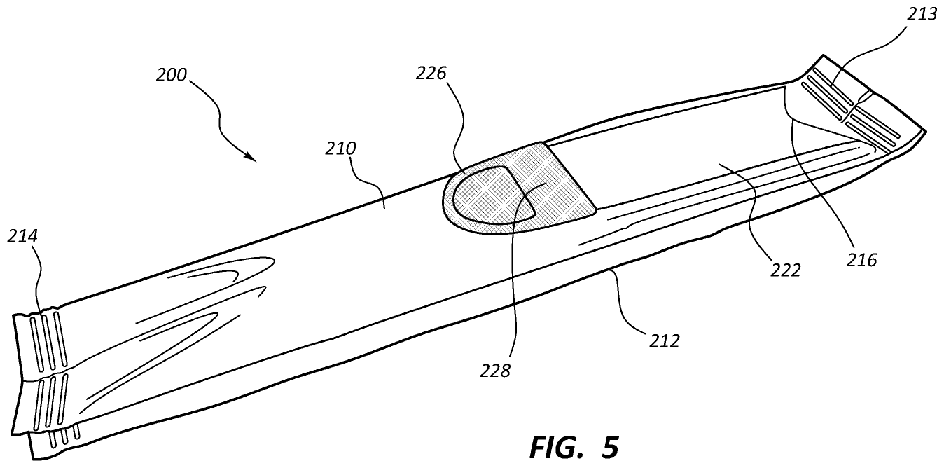

Referring to FIGS. 4a-7, the urinary catheters described herein may be packaged individually in discrete containers to form packaged urinary catheters such as the packaged urinary catheter 200. For example, the packaging or container may be opaque and resemble an item distinct from a urinary catheter, such as a food item or the like. In one embodiment, the packaging or container 210 is formed of and/or includes a foil material. In other embodiments, the container 210 includes a polyolefin film (e.g., polyethylene (PE)), an ethylene vinyl acetate (EVA) film, and/or a metallized polypropylene (PP) film. In one embodiment, the packaging material is gas impermeable. In one embodiment, of the lubricity of the coating is maintained or improved over time in the packaging while at normal environmental storage conditions. The packaging 210 may have a color to indicate sex (e.g., pink for female, blue for male). In one embodiment, the packaging 210 can be sterilized either by Electron Beam Processing (E-beam) or treatment with Ethylene Oxide (EtO).

Referring to FIGS. 4a-4c, a method of manufacturing the packaging for a catheter, discussed herein, including the following steps of producing the package, performed in any order: providing a sheet material 211; providing a weakened area 224, such as a perforation or kiss cut, in the sheet material by cutting the material; folding over and connecting the longitudinal edges 250 of the sheet 211 to form a back seam 212 and a cavity 252 (FIG. 4b). Disposing a catheter 100 within the cavity 252 and enclosed therein by sealing the ends to create a first end seam 213, and a second end seam 214 (FIG. 4c). Adhering an adhesive tab 222 over the weakened area 224.

In one embodiment, this arrangement may be similar to a packaging such as might be used on a candy bar, with overlapping edges forming a seam along the back and seams at the edges. The overlapping edges may be folded to one side or the other. The packaging material may present a smooth front. The front of the container may include a sealed opening 220, covered by an adhesive tab 222 (FIG. 4c). The sealed opening may include a weakened area 224, such as a perforation or kiss cut, in the packaging material covered by an adhesive portion of the adhesive tab 222. The adhesive tab 222 may include features, such as a pull loop 226, to hang the container after exposing the catheter 100 in the packaging in order to facilitate user access to the catheter 100 in the container 210. The adhesive tab 222 may be formed of a material such as polyethylene terephthalate (PET) substrate, with an adhesive, such as an S6 adhesive, on part of or the entire bottom surface of the adhesive tab. In one embodiment, the adhesive tab may include a label. The label may have artwork printed on or otherwise associated with a top surface of the label. The label may be stamped out of a rollstock of material and a varnish may be applied over approximately 1 inch of the distal end 228 of the label to facilitate lifting to begin the peeling process.

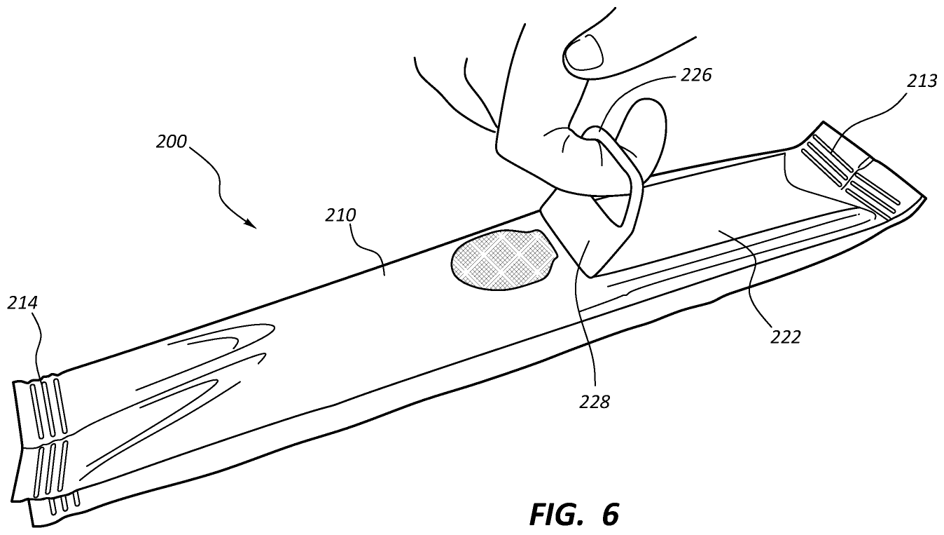

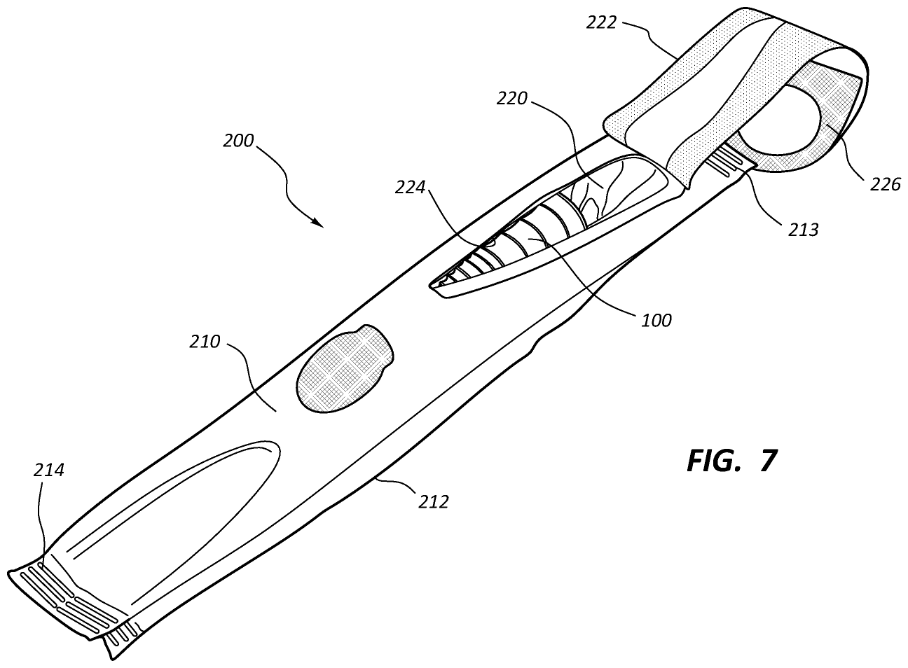

The adhesive tab may include a pull loop 226 to facilitate opening of the container 210, which after opening (FIG. 7) may be positioned over a hook or the like in order to suspend the container for ease of use. Alternatively, the adhesive portion of the adhesive tab 222 may be pressed against a hard surface (e.g., a wall, table, desk, equipment, etc.) in order to prevent movement of the container. In one embodiment, the catheter 100 may be reinserted into the container 210 and the adhesive tab 222 pressed back over the opening 220 to re-seal the container 210 for disposal in another location. The embodiment of FIGS. 4-7 is easy to open by simply putting a finger through the pull loop 226 (FIG. 6) and pulling the adhesive tab toward the proximal end of container 216. The pulling action opens the container along the weakened area 224 to reveal the handle 102 of the catheter 100, which has a gripping surface to facilitate handling. Also, the container can be folded in half to minimize space needed to transport in a purse, bag, or the like.

While the invention has been described in terms of particular variations and illustrative figures, those of ordinary skill in the art will recognize that the invention is not limited to the variations or figures described. Those of ordinary skill in the art will recognize that the invention is not limited to the application of catheters but may be applied to any device that requires similar lubrication. In addition, where methods and steps described above indicate certain events occurring in a certain order, those of ordinary skill in the art will recognize that the ordering of certain steps may be modified and that such modifications are in accordance with the variations of the invention. Additionally, certain of the steps may be performed concurrently in a parallel process when possible, as well as performed sequentially as described above. Further, the features described in one embodiment may generally be combined with features described in other embodiments. Therefore, to the extent there are variations of the invention, which are within the spirit of the disclosure or equivalent to the inventions found in the claims, it is the intent that this patent will cover those variations as well.

What is claimed is:

1. A packaged urinary catheter, comprising:
a urinary catheter including:
a handle;
a catheter shaft attached to the handle;
a base coating over the catheter shaft; and
an outer coating over the base coating, each coating of the base coating and the outer coating including a combination of components selected from a hydrogel, polyacrylic acid (“PAA”), glycerin, water, and polyethylene glycol (“PEG”); and
a container including the urinary catheter, wherein the container does not include an added moisture source or lubricant for application to the catheter shaft, the base coating and the outer coating in a wet state sufficient for insertion of the catheter shaft into a urethra without the added moisture source or lubricant.

2. The urinary catheter of claim 1, wherein the base coating includes PAA in a range of 0.2 wt % to 3 wt %, glycerin in a range of 15 wt % to 25 wt %, water in a range of 20 wt % to 30 wt %, and PEG 400 in a range of 40 wt % to 60 wt %.

3. The urinary catheter of claim 1, wherein the base coating includes PAA in a range of 0.1 wt % to 2.5 wt %, water in a range of 10 wt % to 45 wt %, and PEG in a range of 20 wt % to 65 wt %.

4. The urinary catheter of claim 1, wherein the hydrogel is glyceryl acrylate/acrylic acid copolymer together with glycerin and propylene glycol.

5. The urinary catheter of claim 4, wherein the outer coating includes hydrogel in a range of 20 wt % to 30 wt %, glycerin in a range of 20 wt % to 30 wt %, and PEG 400 in a range of 40 wt % to 60 wt %.

6. The urinary catheter of claim 4, wherein the outer coating includes hydrogel in a range of 20 wt % to 30 wt %, glycerin in a range of 40 wt % to 60 wt %, and PEG 300 in a range of 20 wt % to 30 wt %.

7. The urinary catheter of claim 4, wherein the outer coating includes hydrogel in a range of 10 wt % to 35 wt %, glycerin in a range of 25 wt % to 75 wt %, PEG 300 in a range of 25 wt % to 65 wt %, and PEG 400 in a range of 25 wt % to 50 wt %.

8. The urinary catheter of claim 4, wherein the outer coating includes hydrogel in a range of 15 wt % to 35 wt %, water in a range of 10 wt % to 45 wt %, and PEG 400 in a range of 20 wt % to 75 wt %.

9. The urinary catheter of claim 4, wherein the outer coating includes hydrogel in a range of 22 wt % to 26 wt %, water at approximately 25 wt %, and PEG 400 is in a range of 49 wt % to 53 wt %.

10. The urinary catheter of claim 1, further comprising:
a silicone film over the outer coating, the silicone film configured to be moved toward the handle of the urinary catheter to expose the outer coating before the insertion of the catheter shaft into the urethra.

11. The urinary catheter of claim 10, wherein the silicone film includes ultraviolet (“UV”) light-cured UV agents.

12. The urinary catheter of claim 1, wherein the handle includes a plurality of ridges designed for gripping the handle.

13. The urinary catheter of claim 1, wherein the catheter shaft includes a plurality of eyelets proximate a catheter tip in fluid communication with an opening in a proximal end of the handle by way of a lumen through the urinary catheter.

14. The urinary catheter of claim 1, wherein the plurality of eyelets include two pairs of eyelets circumferentially positioned 90 degrees apart from each other in a non-overlapping configuration.

15. The urinary catheter of claim 1, wherein the container further comprises:
a weakened area of sheet material positioned over at least a portion of the handle of the urinary catheter; and
an adhesive tab covering the weakened area of the sheet material, the adhesive tab including a pull loop configured to open the container about the weakened area of the sheet material when the pull loop is pulled toward an end of the container.

16. A method of manufacturing a packaged urinary catheter, comprising:
folding a sheet material such that longitudinal edges of the sheet material meet;
forming a back seam from the longitudinal edges of the sheet material and, thereby, forming a cavity within the sheet material;
disposing a urinary catheter in the cavity of the sheet material, the urinary catheter including:
a handle;
a catheter shaft attached to the handle;
a base coating over the catheter shaft; and
an outer coating over the base coating, each coating of the base coating and the outer coating including a combination of components selected from a hydrogel, polyacrylic acid (“PAA”), glycerin, water, and polyethylene glycol (“PEG”); and
sealing ends of the cavity of the sheet material to form a container including the urinary catheter, wherein the container does not include an added moisture source or lubricant for application to the catheter shaft, the base coating and the outer coating in a wet state sufficient for insertion of the catheter shaft into a urethra without the added moisture source or lubricant.

17. The method of claim 16, further comprising:
weakening the sheet material before folding the sheet material, thereby providing a weakened area of the sheet material positioned over at least a portion of the handle of the urinary catheter in the container.

18. The method of claim 17, further comprising:
covering the weakened area of the sheet material with an adhesive tab, the adhesive tab including a pull loop configured to open the container about the weakened area of the sheet material when the pull loop is pulled toward an end of the container.

19. The method of claim 16, wherein the base coating includes either:
i) polyacrylic acid (“PAA”) in a range of 0.2 wt % to 3 wt %, glycerin in a range of 15 wt % to 25 wt %, water in a range of 20 wt % to 30 wt %, and polyethylene glycol (“PEG”) 400 in a range of 40 wt % to 60 wt %; or
ii) PAA in a range of 0.1 wt % to 2.5 wt %, water in a range of 10 wt % to 45 wt %, and PEG in a range of 20 wt % to 65 wt %.

20. The method of claim 19, wherein the outer coating includes a combination of components selected from a hydrogel, PAA, glycerin, water, and PEG.
